(19) United States
(12) Patent Application Publication
(10) Pub. No.: US 2003/0165526 A1
(43) Pub. Date: Sep. 4, 2003

(54) Vaccines and agents for inducing immunity against rickettsial diseases, and associated preventative therapy

(76) Inventors: Michael A. Kuzyk, Victoria (CA); Jan Burian, Victoria (CA); William W. Kay, Victoria (CA); Julian C. Thornton, Victoria (CA)

Correspondence Address: KLARQUIST SPARKMAN, LLP, One World Trade Center, 121 S.W. Salmon Street, Suite 1600, Portland, OR, 97204, US

(21) Appl. No.: 10/261,446

(22) Filed: Sep. 30, 2002

Related U.S. Application Data
(63) Continuation of application No. 09/677,374, filed on Sep. 15, 2000.
(60) Provisional application No. 60/154,437, filed on Sep. 17, 1999.

(51) Int. Cl. A61K 39/02

(52) U.S. Cl. 424/190.1

(57) ABSTRACT

The use of the 17 kDa outer surface lipoprotein (OspA) of Piscirickettsia salmonis, or its homologues, as the basis of, or part thereof, a recombinant vaccine for salmonid rickettsial septicaemia and other rickettsial diseases is disclosed. Surface antigens of the bacterial pathogen P. salmonis are characterized and an immunoreactive antigen, namely the 17 kDa outer surface lipoprotein OspA of P. salmonis, as well as the nucleic acid segment that encodes the OspA immunoreactive antigen, is identified and characterized. Diagnostic techniques including the use of hybridization probes and primers as well as the production of specific antigens and antibodies that may be used in immunization techniques for inducing immunity against P. salmonis and other rickettsial diseases are disclosed, as are the development of recombinant vaccines for SRS and other rickettsial diseases based on the 17 kDa lipoprotein OspA. Augmentation of protective immunity by the inclusion of promiscuous T lymphocyte epitopes (TCE's) in fusion protein constructs in salmonids and to the use of bacterial protein inclusion bodies as a source of the protective immunogen is also disclosed.

### CROSS REFERENCE TO RELATED APPLICATIONS

This is a continuation of application Ser. No. 09/667,374, filed Sep. 15, 2000, which claims the benefit of U.S. Provisional Application No. 60/154,437, filed Sep. 17, 1999, each of which prior applications is incorporated herein by reference.

### FIELD OF THE INVENTION

This invention relates generally to the field of obligate intracellular bacteria, and in particular to agents of rickettsia type diseases, specifically Piscirickettsia salmonis in aquatic poikilotherms. The invention also encompasses isolated genes encoding outer surface antigens of P. salmonis and the diagnostic and therapeutic use (including in particular the preparation of a recombinant vaccine to prevent or reduce the incidence of infection by P. salmonis and other rickettsial diseases) of such antigens or their homologues.

In particular aspects, this invention relates to the use of the 17 kDa outer surface lipoprotein (OspA) of Piscirickettsia salmonis, or its homologues, as the basis of, or part thereof, a recombinant vaccine for salmonid rickettsial septicaemia and other rickettsial diseases. This invention also relates to the augmentation of protective immunity by the inclusion of promiscuous T lymphocyte epitopes (TCE's) in fusion protein constructs in salmonids. This invention also relates to the use of bacterial protein inclusion bodies as a source of the protective immunogen. The applicant recognizes that some of the foregoing inventive concepts are sufficiently distinct from one another that the set of inventive concepts may have to be protected in more than one patent.

### BACKGROUND OF THE INVENTION

The order Rickettsiales historically encompassed any intracellular bacterium and taxonomy was based on only a few phenotypic characteristics (Drancourt and Raoult, 1994). More recently, 16S rRNA sequence similarity studies have helped to better define the taxonomy of the order Rickettsiales (Drancourt and Raoult, 1994). Rickettsiae cause a variety of medically significant diseases in humans including typhus fever, Rocky Mountain spotted fever, and boutonneuse fever (Pang and Winkler, 1994; Vishwanath, et al., 1990). Rickettsiae are also agriculturally significant, and are the aetiological agents of a variety of veterinary diseases (Rikihisa, 1991).

The past decade has been a renaissance in the identification of rickettsial and rickettsial-like infections as the aetiological agents of poorly understood diseases and as emerging pathogens (Anderson, 1997; Azad, et al., 1997; Davis, et al., 1998; Fryer and Mauel, 1997; Stenos, et al., 1998). Inherent difficulties are associated with rickettsials: it is very difficult to grow large quantities of rickettsiae; rickettsiae have very slow growth rates; and rickettsiae are difficult to separate from host cell material (Higgins, et al., 1998). Although rickettsiae lack a characterized genetic system for genetic manipulation (Mallavia, 1991), the advent of recombinant DNA technology has revolutionized rickettsial research. Characterization of rickettsial pathogenesis and functional analysis of rickettsial antigens has largely relied upon antibody inactivation studies (Li and Walker, 1998; Messick and Rikihisa, 1994; Seong, et al., 1997). Recently major rickettsial antigens have been identified and characterized further upon sub-cloning into Escherichia coli (Anderson, et al., 1990; Anderson, et al., 1987; Carl, et al., 1990; Ching, et al., 1992; Ching, et al., 1996; Hahn and Chang, 1996; Musoke, et al., 1996). Successful transformation of Rickettsia typhi (Troyer, et al., 1999) and Rickettsia prowazekii (Rachek, et al., 1998) have recently raised exciting prospects for the future of rickettsia research.

Antibody studies of rickettsiae have shown that inactivation of specific rickettsial surface proteins can inhibit entry into host cells and establishment of infection (Anacker, et al., 1985; Li and Walker, 1998; Messick and Rikihisa, 1994). Failed attempts at constructing vaccines against human rickettsial diseases have been based on preparations of inactivated whole cells (Sumner, et al., 1995). Although these whole cell vaccines elicit protective responses in animal models, they are only partially effective when used in humans (Sumner, et al., 1995). Current vaccine strategies using recombinantly expressed rickettsial proteins identified by antibody studies have been shown to successfully elicit protective immune responses against bacterial challenge (McDonald, et al., 1987; Sumner, et al., 1995).

Piscirickettsia salmonis is the first rickettsiae to be isolated from an aquatic poikilotherm (Fryer, et al., 1990). P. salmonis is the aetiological agent of salmonid rickettsial septicaemia (SRS), and is an economically significant pathogen of salmonids that is responsible for extensive mortalities in the cold water aquaculture industry. P. salmonis, a gram-negative obligate intracellular bacterium, was first observed in 1989 in a diseased, moribund coho salmon from a saltwater net pen site on the coast of Chile (Bravo and Campos, 1989). It is now known that P. salmonis is geographically more widespread than was initially suspected, and has recently been observed in Ireland (Rodger and Drinan, 1993), Scotland, Norway, and on the Pacific coast of Canada (Brocklebank, et al., 1993).

P. salmonis has been observed to infect a wide range of salmonid species and causes a systemic infection that targets the kidney, liver, spleen, heart, brain, intestine, ovary, and gills of salmonids (Cvitanich, et al., 1991). Pleomorphic, predominantly coccoid bacteria that range in diameter from 0.5 to 1.5 μm are found within cytoplasmic vacuoles of cells from infected tissues (Bravo and Campos, 1989). While initially difficult to culture, P. salmonis was successfully isolated from the kidney of a diseased adult coho salmon on an immortal chinook salmon embryo cell line (Fryer, et al., 1990). Fryer et al. (Fryer, et al., 1992) conducted a 16S rRNA sequence similarity study which placed P. salmonis in its own genus and species within the order Rickettsiales. P. salmonis is most closely related to Coxiella burnetii and Wolbachia persica with 87.5% and 86.3% sequence similarity respectively (Fryer, et al., 1992). P. salmonis appears to belong within the tribe Ehrlichieae because of its morphological characteristics (Fryer, et al., 1992).

Efficacy of antibiotic treatment of SRS is poor because of the intracellular nature of P. salmonis, thereby making management of the disease difficult (Lannan and Fryer, 1993). To effectively prevent and control SRS, vaccine development is desirable. However, vaccines prepared from whole cell bacterins of mammalian rickettsiae have shown disappointing protection in trials (Hickman, et al., 1991).

Incorporation of highly immunogenic T lymphocyte epitopes (TCE's) into chimeric fusion proteins is an elegant extension of the principles that underlie the immunostimulatory effect of toxoid carrier proteins on conjugated haptens (Bixler and Pillai, 1989). Toxoids provide TCE's that are required to elicit a strong T helper cell-mediated immune response against haptens (Bixler and Pillai, 1989). Incorporation of TCE's into synthetic peptide or chimeric fusion proteins can have an immunostimulatory effect on other T cell and humoral epitopes within the peptide or protein (Hathaway, et al., 1995; Kjerrulf, et al., 1997; O'Hem, et al, 1997; Pillai, et al, 1995; Valmori, et al., 1992). To minimize genetic restriction of these immunostimulatory responses, promiscuous TCE's capable of binding major histocompatibility complex (MHC) molecules from a variety of haplotypes are used in chimeric vaccine constructs. Tandem repeats of TCE's can also often improve immunogenicity of chimeric proteins better than single TCE's (Kjerrulf, et al., 1997; Partidos, et al., 1992).

The Clostridium tetani tetanus toxin P2 (tt P2) and measles virus fusion protein (MVF) epitopes have been established as strong TCE's that exhibit promiscuous binding to various MHC haplotypes and are highly immunogenic in human and murine models (Demotz, et al., 1989; Panina-Bordignon, et al., 1989; Partidos and Steward, 1990). Both tt P2 and MVF TCE's are MHC class II restricted and are able to bind MHC class II molecules from a wide variety of haplotypes. Genetic restriction of murine responses to malarial epitopes has been overcome by incorporation of the tt P2 epitope into synthetic peptide-based malarial vaccines (Valmori, et al., 1992).

### SUMMARY OF THE INVENTION

The present inventors have characterized the surface antigens of the bacterial pathogen P. salmonis and identified and characterized an immunoreactive antigen, namely the 17 kDa outer surface lipoprotein OspA of P. salmonis, as well as the nucleic acid segment that encodes the OspA immunoreactive antigen. This discovery enables the development of diagnostic techniques (including the use of hybridization probes and primers) as well as the production of specific antigens and antibodies that may be used in immunization techniques for inducing immunity against P. salmonis and other rickettsial diseases. In particular, the discovery enables the development of recombinant vaccines for SRS and other rickettsial diseases based on the 17 kDa lipoprotein OspA.

In one embodiment, the invention comprises an isolated nucleic acid segment (SEQ ID NO:1) encoding a 17 kDa immunodominant protein of P. salmonis, which is immunoreactive with anti-P. salmonis serum. In another embodiment, the invention comprises a nucleic acid segment that encodes a protein having the amino acid sequence of SEQ ID NO:2, including variants that retain immunogenicity. Due to the degeneracy of the genetic code and the possible presence of flanking nucleic acid fragments outside of the coding region, it will be understood that many different nucleic acid sequences may encode the amino acid sequence of SEQ ID NO:2 and variants, and that all such sequences would be encompassed within the present invention.

The nucleic acid segment of the invention may be modified for optimal codon usage and expression in a host cell line (i.e. “optimized”) as shown, for example, in SEQ ID NO:3, and may be operably linked to a recombinant promoter and a TCE fusion partner as, for example, in SEQ ID NO:5.

In a further embodiment, the invention relates to the use of OspA as an immunogen and to the use of OspA in a recombinant vaccine to reduce the incidence of infection by P. salmonis and other rickettsial diseases.

The slow growing, rickettsia-like, piscine pathogen, P. salmonis, was grown en mass on chinook salmon (Oncorhynchus tshawytscha) embyro cell line monolayers (CHSE-214) to purify enough P. salmonis to allow genomic deoxyribonucleic acid (DNA) isolation. A genomic expression library was constructed and screened with high titre anti-P. salmonis rabbit serum identifying immunoreactive clones that encoded a common region of P. salmonis DNA. A 4,983 bp insert was excised in E. coli and Exo III/S1 deletion clones were sequenced. The insert contained 4 intact open reading frames (ORF) one of which encoded a homologue, ospA, of a genus-specific, rickettsia-like, outer membrane 17 kDa lipoprotein antigen. OspA was recognized by both convalescent coho salmon (Oncorhynchus kisutch) serum and rabbit antiserum to both 10 & 20 residue peptides based on predicted protein sequence. The codon usage of the ospA ORF was optimized for expression in E. coli by construction of a synthetic version of the ospA gene. An N-terminal fusion partner was cloned in frame with the ospA gene as well as tt P2 and MVF TCE's all under the control of both T7 and lambda phage promoters to direct expression into inclusion bodies as well as to facilitate large scale expression of the protein. The various OspA fusion proteins were purified from E. coli as the insoluble inclusion body fraction of a whole cell lysate. Suspensions of the insoluble fraction were formulated with an adjuvant and used as a vaccine to immunize coho salmon. Vaccinates showed both an increase in anti-OspA antibody production and increased in vitro stimulation of whole lymphocyte populations by OspA fusion protein. Eight weeks post-vaccination, the salmon were challenged with virulent suspensions of P. salmonis. The results indicated that the vaccine was protective against virulent challenge and that immunogenicity and protection were augmented by the incorporation of promiscuous TCE's into the OspA fusion protein.

Functional presentation of antigen by salmonid MHC class I and II complexes analogous to the role of MHC class I and II of mammals and birds has not been confirmed in teleosts. As a result, algorithms do not exist for predicting peptide sequences that are capable of functioning as TCE's in the salmonid immune system. As tt P2 and MVF epitopes have been established as strong epitopes that exhibit promiscuous binding to various MHC haplotypes, these epitopes were incorporated onto the OspA fusion protein to elicit immunostimulatory effects. Although the incorporation of highly immunogenic promiscuous TCE's into chimeric fusion proteins to extend the immunostimulatory effect of toxoid carrier proteins on conjugated haptens is not per se novel, the immunostimulating effects of TCE's within the salmonid immune system is novel. Furthermore, the novelty of the immunostimulating effects of TCE's within teleosts is not dependent upon the identification and characterization of the outer surface lipoprotein OspA of P. Salomonis.

Sequence Listing

The nucleic and amino acid sequences listed in the accompanying sequence listing are shown using standard letter abbreviations for nucleotide bases, and one letter code for amino acids. Only one strand of each nucleic acid sequence is shown, but the complementary strand is understood as included by any reference to the displayed strand.

| | |
|---|---|
| SEQ ID:1 | shows the ospA DNA sequence from P. salmonis |
| SEQ ID:2 | shows the amino acid sequence of the precursor |
| | (unprocessed) protein OspA |
| SEQ ID:3 | shows the ospA DNA sequence, 17e2, modified for optimal |
| | codon usage in E. coli |
| SEQ ID:4 | shows the amino acid sequence of the modified for optimal |
| | codon usage, in E. coli, precursor (unprocessed) protein |
| | OspA (17E2) |
| SEQ ID:5 | shows the DNA sequence, c17e2, of an N-terminal fusion |
| | partner with optimized ospA gene |
| SEQ ID: 6 | shows the amino acid sequence of an N-terminal fusion |
| | partner with optimized OspA (C17E2) |
| SEQ ID:7 | DNA sequence of the forward oligonucleotide used during |
| | pTYB1-17kDa construction |
| SEQ ID:8 | DNA sequence of the reverse oligonucleotide used during |
| | pTYB1-17kDa construction |
| SEQ ID:9 | oligonucleotide #1 used for construction of optimized |
| | ospA gene, 17e2 |
| SEQ ID:10 | oligonucleotide #2 used for construction of optimized |
| | ospA gene, 17e2 |
| SEQ ID:11 | oligonucleotide #3 used for construction of optimized |
| | ospA gene, 17e2 |
| SEQ ID:12 | oligonucleotide #4 used for construction of optimized |
| | ospA gene, 17e2 |
| SEQ ID:13 | oligonucleotide #5 used for construction of optimized |
| | ospA gene, 17e2 |
| SEQ ID:14 | oligonucleotide #6 used for construction of optimized |
| | ospA gene, 17e2 |
| SEQ ID:15 | amino acid sequence of a 10 residue synthetic polypeptide |
| | based on residues 110-119 of OspA |
| SEQ ID:16 | amino acid sequence of a 20 residue synthetic polypeptide |
| | based on residues 110-129 of OspA |
| SEQ ID:17 | DNA sequence of the tt P2 TCE oligonucleotide |
| SEQ ID:18 | DNA sequence of the MVF TCE oligonucleotide |
| SEQ ID:19 | amino acid sequence of the tt P2 TCE |
| SEQ ID:20 | amino acid sequence of the MVF TCE |

### BRIEF DESCRIPTION OF THE DRAWINGS

FIG. 1. Western blot analysis of P. salmonis. Whole cell lysate and proteinase K digest samples of P. salmonis were separated by 12% SDS-PAGE and reacted with rabbit anti-P. salmonis polyclonal antibodies followed by immunochemical detection. Note the immunoreactive protein migrating at 17 kDa. The ˜11 kDa antigen of P. salmonis was not susceptible to PK digestion. Molecular weights are in kDa.

FIG. 2. A. Schematic of spatial relationships of ORF's in P. salmonis clone pB12, 4,983 bp. The Xba I and Hind III sites were used to subclone the ospA ORF into pBC(+) (Example 2). B. DNA sequence of the P. salmonis ospA ORF and amino acid sequence of the OspA protein translated from the ospA ORF. C. Pairwise sequence alignment of the P. salmonis 17 kDa antigen, OspA, and the R. prowazekii 17 kDa antigen (SwissProt G112704). The pairwise alignment was generated using the FASTA3 algorithm. The P. salmonis 17 kDa antigen shares 41% identity (black background) and 62% similarity (black box) with the 17 kDa antigen of R. prowazekii. Synthetic peptides (SEQ ID:15, SEQ ID:16) representing the region from residues 110-129 of the P. salmonis 17 kDa antigen were used to generate rabbit polyclonal serum.

FIG. 3. A. Map of pBC-17 kDa, the pBC(+) plasmid encoding the subcloned ospA ORF (Xba I/Hind III fragment of clone pB 12). Cm is chloramphenicol resistance, T7 is T7 promoter. B. Analysis of OspA expression. Whole cell lysates of E. coli clones and P. salmonis were analyzed by SDS-PAGE (12% polyacrylamide). P. salmonis whole cell lysate was reacted with rabbit polyclonal serum generated against a 10 residue peptide (SEQ ID:15) of OspA recognizing a strongly immunoreactive product in the 17 kDa region of P. salmonis. Expression of the OspA by clone pBC-17 kDa was induced at 42° C. and is visible stained by Coomassie blue. Rabbit polyclonal serum generated against a 20 residue peptide (SEQ ID:16) of OspA recognized the expressed 17 kDa protein in induced pBC-17 kDa samples. Convalescent serum from coho salmon also recognized the induced 17 kDa protein in pBC-17 kDa. Arrows identify the expressed 17 kDa antigen. Molecular weight standards are shown in kDa.

FIG. 4. A. Schematic representation of the strategy employed during the synthesis of the E. coli codon optimized ospA gene, 17e2. B. DNA sequence of the 6 overlapping oligonucleotides used. C. DNA sequence of the E. coli codon optimized ospA gene, 17e2.

FIG. 5. A. Amino acid sequence of the OspA protein, 17E2, expressed from the optimized ospA gene, 17e2. B. DNA sequence of the N-terminal ospA gene fusion construct, c17e2. C. Amino acid sequence of the OspA-fusion protein, C17E2, containing an N-terminal fusion.

FIG. 6. A. Maps of the expression vectors encoding the optimized ospA fusion construct under the control of T7, pETC-17E2, and lambda promoters, pKLPR-C17E2. Ap is ampicillin resistance, Km is kanamycin resistance, T7 P is the T7 promoter, PLR is lambda right promoter. B. 12% polyacrylamide SDS-PAGE analysis of C17E2 expression. Samples from the lambda promoter expression represent the insoluble fraction (i.f.) of whole cells lysates. Whole cell (w.c.) samples from T7 expression are loaded along with a sample of the insoluble fraction Note the abundant expression of the OspA-fusion product at 28.5 kDa in the induced samples. Molecular weight standards are shown in kDa.

FIG. 7. Map of pTYB1-17 kDa. An ospA-fusion construct encoding a C-terminal fusion partner was placed under the control of T7 promoter. The C-terminal fusion partner contained a self-cleaving spacer region and chitin binding domain.

FIG. 8. A. A diagram illustrating the cloning strategy employed to create the OspA fusion protein constructs encoding promiscuous TCE's. 17E2 is the synthetic ospA gene that was created using codons optimized for E. coli high level expression. tt P2 and MVF are the DNA sequences (SEQ ID:19, SEQ ID:20) encoding the tetanus toxin and measles virus fusion protein T cell epitopes (SEQ ID:17, SEQ ID:18). B. (a) Sequences of the tt P2 and (b) MVF oligonucleotides (SEQ ID:17, SEQ ID:18) used to incorporate the (c) tt P2 and (d) MVF TCE's (SEQ ID:19, SEQ ID:20) into the OspA fusion protein constructs. Bold nucleotides indicate the TCE coding region of the oligonucleotides.

FIG. 9. Antibody titres of coho salmon groups against OspA-fusion protein candidate vaccines. Salmon were immunized with either C17E2, CT17E2, CM17E2, or CMT17E2. Antibody titres were defined as the maximum serum dilution that resulted in a signal corresponding to 3 times the background obtained with the diluent vaccinated serum group at a dilution of 1:320.

FIG. 10. Proliferative lymphocyte responses of vaccinated Atlantic salmon (Salmo salar). The highest lymphocyte stimulation occurred in salmon that were vaccinated with an OspA fusion protein containing two promiscuous TCE's (CMT17E2).

FIG. 11. Vaccine trial of OspA fusion protein constructs containing promiscuous TCE's in an outbred population of coho salmon. Adjuvant-injected salmon experienced a cumulative mortality of 85.5% when challenged with P. salmonis by IP injection. C17E2 vaccinated salmon reached a cumulative mortality of 59.6%. CT17E2 vaccinated salmon experienced 35.6% cumulative mortality. CM17E2 and the CM17E2+CT17E2 groups experienced 20 and 18.6% cumulative mortality, respectively. The CMT17E2 vaccinated group experienced only 14.5% cumulative mortality. RPS values of C17E2, CT17E2, CM17E2, CM17E2 +CT17E2, and CMT17E2 were 30.2, 58.4, 76.6, and 83.0%, respectively.

### DETAILED DESCRIPTION OF THE INVENTION

I. Definitions

Epitope: An epitope refers to an immunologically active region of an immunogen (most often a protein, but sometimes also a polysaccharide or lipid) that binds to specific membrane receptors for antigen on lymphocytes or to secreted antibodies. To generate an immune response to a foreign antigen, lymphocytes and antibodies recognize these specific regions (epitopes) of the antigen rather than the entire molecule.

B cell epitope: The region (epitope) of an immunogen which is recognized by B cells when it binds to their membrane bound antibody. The B cells which recognize that particular region then proliferate and secrete antibody molecules which are specific for that region of the immunogen. B cell epitopes tend to be highly accessible regions on the exposed surface of the immunogen. Stimulation of the immune system by B cell epitopes results in “humoral” immunity.

T cell epitope: The region (epitope) of an immunogen which is recognized by a receptor on T cells after being processed and presented on the surface of an antigen presenting cell (APC) in the context of a major histocompatability complex (MHC) class I or II molecule. T cells can be split into two distinct groups, T helper cells (Tₕ) and T cytotoxic cells (T^{c}). T helper cells recognize epitopes bound to MHC class II molecules whereas T cytotoxic cells recognize epitopes bound to MHC class I molecules. T helper cells can be further subdivided into two classes, Tₕ₁, and Tₕ₂, Tₕ₁being responsible for stimulation of cell-mediated immunity and Tₕ₂ cells stimulating the humoral arm of the immune system. When a given T cell recognizes the epitope-MHC complex at the surface of the APC it becomes stimulated and proliferates, leading to the production of a large number of T cells with receptors specific for the stimulating epitope. Stimulation of the immune system by T cell epitopes normally results in “cell-mediated” immunity.

Attenuated Bacterial Vaccine: This refers to bacterial strains which have lost their pathogenicity while retaining their capacity for transient growth within an inoculated host. Because of their capacity for transient growth, such vaccines provide prolonged immune-system exposure to the individual epitopes on the attenuated organisms, resulting in increased immunogenicity and memory-cell production, which sometimes eliminates the need for repeated booster injections. The ability of many attenuated vaccines to replicate within host cells makes them very suitable to induce a cell-mediated immunity. Typically, bacterial strains are made attenuated by introducing multiple defined gene mutations into the chromosome thereby impairing growth in vivo.

Recombinant Vector Vaccine: This refers to the introduction of genes (or pieces of genes) encoding major antigens (or epitopes) from especially virulent pathogens into attenuated viruses or bacteria. The attenuated organism serves as a vector, replicating within the host and expressing the gene product of the pathogen.

Sequence Identity: Identity between two nucleic acid sequences, or two amino acid sequences is expressed in terms of the level of identical residues shared between the sequences. Sequence identity is typically expressed in terms of percentage identity; the higher the percentage, the more similar the two sequences are.

Sequence Similarity: Similarity between two amino acid sequences is expressed in terms of the level of sequence conservation, including shared identical residues and those residues which differ but which share a similar size, polarity, charge or hydrophobicity. Sequence similarity is typically expressed in terms of percentage similarity; the higher the percentage, the more similar the two sequences are.

Recombinant: A recombinant nucleic acid is one that has a sequence that is not normally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques.

Oligonucleotide (oligo): A linear polymer sequence of up to approximately 100 nucleotide bases in length.

Probes and primers: Nucleic acid probes and primers may readily be prepared based on the amino acid and DNA sequence provided by this invention. A probe comprises an isolated nucleic acid attached to a detectable label or reporter molecule. Typical labels include radioactive isotopes, ligands, chemiluminescent agents, and enzymes. Methods for labeling and guidance in the choice of labels appropriate for various purposes are discussed, e.g., in Sambrook et al.

Primers are short nucleic acids, preferably DNA oligonucleotides 15 nucleotides or more in length. Primers may be annealed to a complementary target DNA strand, and then extended along the target DNA strand by a DNA polymerase enzyme. Primer pairs can be used for amplification of a nucleic acid sequence, e.g., by the polymerase chain reaction (PCR) or other nucleic-acid amplification methods known in the art.

Methods for preparing and using probes and primers are described, for example, in Sambrook, 1989, Ausubel, 1987, and Innis, 1990. PCR primer pairs can be derived from a known sequence, for example, by using computer programs intended for that purpose such as DNAStar Lasergene software. One of skill in the art will appreciate that the specificity of a particular probe or primer increases with its length. Thus, for example, a primer comprising 20 consecutive nucleotides will anneal to a target with a higher specificity than a corresponding primer of only 15 nucleotides. Thus, in order to obtain greater specificity, probes and primers may be selected that comprise 20, 25, 30, 35, 40, 50 or more consecutive nucleotides.

Isolated: An “isolated” biological component (such as nucleic acid or protein or organelle) has been substantially separated or purified away from other biological components in the cell of the organism in which the component naturally occurs, i.e., other chromosomal and extra-chromosomal DNA and RNA, proteins and organdies. Nucleic acids and proteins that have been “isolated” include nucleic acids and proteins purified by standard purification methods. The term also embraces nucleic acids and proteins prepared by recombinant expression in a host cell as well as chemically synthesized nucleic acids. An “isolated” bacterial strain or colony is purified away from other colonies and yields a pure culture without any contaminants upon plating on selective media.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art. A “temperature-sensitive” vector is one which replicates normally at a low growth temperature (i.e., 28 C.) and will not replicate at a higher growth temperature (i.e., 42 C.) due to mutations at or near the origin of replication. An “imperfectly segregating” vector is one which is not stably inherited by new daughter cells at the time of cell division in the absence of selection pressure due to mutations within the vector sequence.

Host Cell: Refers to those cells capable of growth in culture and capable of expressing OspA protein and/or OspA fusion protein. The host cells of the present invention encompass cells in in vitro culture and include prokaryotic and eukaryotic, including insect cells. A host cell strain may be chosen which modulates the expression of the inserted sequences, or modifies and processes the gene product in the specific fashion desired. Expression from certain promoters can be elevated in the presence of certain inducers (i.e. temperature, small inducer molecules such as -galactosides for controlling expression of T7 or lac promoters or variants thereof). The preferred host cell for the cloning and expression of the OspA protein and OspA-fusion protein is a prokaryotic cell. An example of a prokaryotic cell useful for cloning and expression of the OspA protein of the present invention is E. coli BL21.

Cell Culture: a) Refers to the growth of eukaryotic (non-bacterial) cells in a complex culture medium generally consisting of vitamins, buffers, salts, animal serum, and other nutrients. (b) Refers to the growth of P. salmonis on CHSE-214 and any other cell line that sustains P. salmonis growth.

Fusion Partner: Any DNA sequence cloned in frame to the 5′ or 3′ end of an ORF that results in transcription and translation of amino acid sequence added to the N- or C-terminus of the original protein.

Fusion Protein: The term fusion protein used herein refers to the joining together of at least two proteins, an OspA protein and a second protein. In some embodiments of the present invention, the second protein may be fused or joined to a third protein. In the present invention, examples of second proteins include any polypeptide that facilitates the following: expression, secretion, purification, condensation, precipitation, or any property which facilitates concentration or purification.

Variant: Any molecule in which the amino acid sequence, glycosylation, phosphorylation, and/or lipidation pattern, or any other feature of a naturally occurring molecule which has been modified covalently or non-covalently and is intended to include mutants. Some of the variants falling within this invention possess amino acid substitutions, deletions, and/or insertions provided that the final construct possesses the desired ability of OspA. Amino acid substitutions in OspA may be made on a basis of similarity in polarity, charge, solubility, hydrophobicity, hydrophilicity, and/or the amphipathic nature of the residues involved. Also included within the definition of variant are those proteins having additional amino acids at one or more of the C-terminal, N-terminal, and within the naturally occurring OspA sequence as long as the variant protein retains the desired capability of OspA to act as an antigen and hence as a vaccine.

| Original Residue | Conservative Substitutions |
|---|---|
| Ala | ser |
| Arg | lys |
| Asn | gln; his |
| Asp | glu |
| Gln | asn |
| Glu | asp |
| Gly | pro |
| His | asn; gln |
| Ile | leu; val |
| Leu | ile; val |
| Lys | arg; gln; glu |
| Met | leu; ile |
| Phe | met; leu; tyr |
| Ser | thr |
| Thr | ser |
| Trp | tyr |
| Tyr | trp; phe |
| Val | ile; leu |

Table 1: More substantial changes in functional or other features may be obtained by selecting substitutions that are less conservative than those in Table 1, i.e., selecting residues that differ more significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. The substitutions which in general are expected to produce the greatest changes in protein properties will be those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine. Variant proteins having one or more of these more substantial changes may also be employed in the invention, provided that immunogenicity of OspA is retained.

More extensive amino acid changes may also be engineered into variant OspA. As noted above however, these variants will typically be characterized by possession of at least 40% sequence identity counted over the full length alignment with the amino acid sequence of their respective naturally occurring sequences using the alignment programs described herein. In addition, these variant OspA proteins would retain immunogenicity.

Confirmation that OspA has immunogenic activity may be achieved using the immunological and protection experiments described herein. Following confirmation that OspA has the desired immunogenic effect, a nucleic acid molecule encoding OspA may be readily produced using standard molecular biology techniques. Where appropriate, the selection of the open reading frame will take into account codon usage bias of the bacterial or eukaryotic species in which OspA is to be expressed.

Inclusion body: Intracellularly confined, insoluble, protein-containing particles of bacterial cells comprised of either homologous or heterologous proteins. These particles are the reservoirs and consequence of overproduction of bacterial recombinant proteins. Inclusion bodies can be purified or semi-purified and used directly as protein antigens or can be solubilized by various procedures and used as soluble protein antigen preparations.

Alignment programs: Methods for aligning sequences for comparison purposes are well known in the art. Various programs and alignment algorithms are described in Smith and Waterman (1981), Needleman and Wunsch (1970), Pearson and Lipman (1988), Higgins and Sharp (1988, 1989), Corpet et al (1988), Huang et al. (1992), Pearson et al. (1994). Altschul et al. (1990) presents a detailed consideration of sequence alignment methods.

The National Centre of Biotechnology Information (NCBI) Basic Local Alignment Search Tool (BLAST; Altschul et al., 1990) is available from several sources, including the NCBI (Bethesda, Md.) and on the Internet, for use in connection with the sequence analysis programs BLASTP, BLASTN, BLASTX, TBLASTN, TBLASTX. BLAST can be accessed at http://www.ncbi.nlm.nih.gov/BLAST/. A description of how to determine sequence identity using this program is available at http ://www.ncbi.nlm.nih.gov/BLAST/blast_help.html.

For comparisons of amino acid sequences of greater than 30 amino acids, the “BLAST 2 Sequences” function in the BLAST program is employed using the BLASTP program with the default BLOSUM62 matrix set to default parameters, (open gap 11, extension gap 1 penalties). When aligning short peptides (fewer than 30 amino acids), the alignment should be performed using the “Blast 2 Sequences” function employing the BLASTP program with the PAM30 matrix set to default parameters (open gap 9, extension gap 1 penalties). Proteins having even greater similarity to the reference sequences will show increasing percentage identities when assessed by this method, such as at least 45%, at least 50%, at least 60%, at least 70%, at least 75%, at least 80%, at least 85%, at least 90%, or at least 95% sequence identity.

Promoter: A region of DNA to which either RNA polymerase or any other enhancer protein binds before initiating transcription of the DNA code into the RNA gene product. For example; lambda, phage T7, lac, tac, srpP, trpP, or araB etc. promoter DNA. A promoter region therefore determines the efficiency of the RNA gene product.

Fragments: Those parts of either the DNA encoding a gene for a protein, a TCE, or a fusion partner and those parts of the protein, TCE, or fusion partner itself.

II. Selection and Creation of Nucleic Acid Sequences Encoding the 17 kDa OspA Protein

a. Growth & Purification of P. salmonis

P. salmonis strains were routinely passaged on chinook salmon embryo cell line CHSE-214 (ATCC CRL-1681) at 17° C. in Eagle's minimal essential media (MEM) with Earle's salts supplemented with 10% newborn calf serum. Type strain P. salmonis LF-89 was obtained from the American Type Culture Collection (ATCC VR-1361) and is herein referred to as P. salmonis.

A protocol for purifying P. salmonis was developed by combining and modifying the protocols of Tamura et al (Tamura, et al., 1982) and Weiss et al (Weiss, et al, 1975). A 6,320 cm2 Nunc cell factory was seeded with cell line CHSE-214 and infected with 450 ml of cell culture supernatant from fully lysed CHSE-214 monolayers infected with P. salmonis. Infection was allowed to continue 14-17 days until cytopathic effects obliterated the entire monolayer. Upon destruction of the monolayers cell culture supernatants were collected and centrifuged at 10,000×g for 30 min at 4° C. Pellets were resuspended in MEM and homogenized in a 15 ml Dounce tissue homogenizer.

The homogenized suspension was centrifuged at 200×g for 10 min at 4° C. to pellet large host cell debris. The supernatant was filtered twice through glass microfibre and centrifuged at 17,600×g for 15 min at 4° C. Pellets were resuspended in TS-buffer (33 mM Tris-HCl, 0.25 M sucrose; pH 7.4). Samples were loaded onto Percoll gradients with a final concentration of 40% and centrifuged in a fixed angle rotor (type JA-14) at 20,000×g for 60 min at 4° C. in a Beckman J2-21 centrifuge. Bands were collected by aspiration, diluted with phosphate buffered saline, pH 7.4 (Sambrook, et al., 1989) and centrifuged at 20,000×g for 10 min at 4° C. Pellets were washed twice with phosphate buffer solution (PBS). Contents of the bands were negative stained with 0.5% phosphotungstic acid and analyzed by transmission electron microscopy on a Phillips EM 300 at an accelerating voltage of 75 kV.

b. Demonstration of Immunoreactive Molecules

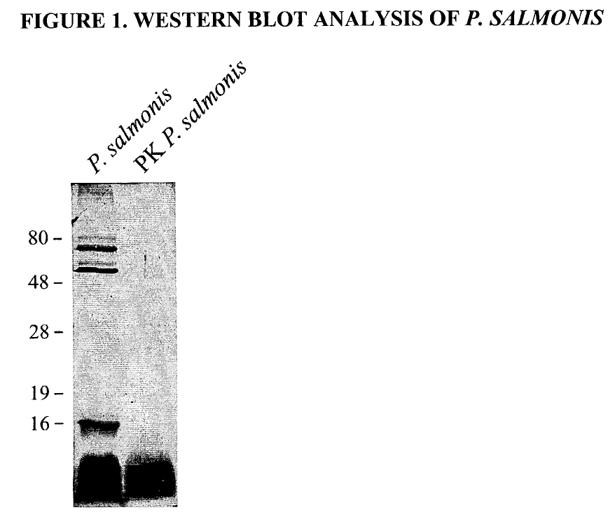

In order to characterize the antigenic profile of P. salmonis, western blot analysis was carried out using anti-P. salmonis rabbit serum (FIG. 1). Proteinase K digestion was used to determine if any observed antigens may have been carbohydrate. Six P. salmonis immunoreactive antigens were observed at relative molecular weights of 65, 60, 54, 51, 17, and 11 kDa (FIG. 1). Proteinase K digestion destroyed all immunoreactive antigens except the 11 kDa antigen (FIG. 1).

c. Purification of Genomic DNA & Construction of Library

P. salmonis was purified by density gradient centrifugation as previously described (Kuzyk, et al., 1996) from 12,000 cm2 of CHSE-214 cells exhibiting full cytopathic effect 14 days after infection with P. salmonis. A single step DNA isolation solution was used to obtain genomic DNA from the purified P. salmonis. Genomic DNA was further purified by equilibrium centrifugation using a CsCl-ethidium bromide gradient to yield 250 μg of P. salmonis genomic DNA (Sambrook, et al., 1989). P. salmonis DNA was partially digested using serially diluted EcoR I. Digests containing an average fragment size of 10 kb were chosen for creation of a P. salmonis gene expression library using a lambda ZAP II cloning kit.

d. Immunological Screening of Library

Approximately 10,000 plaques of P. salmonis lambda expression library were screened per round with a desired density of 1,000 plaques per 80 mm petri dish. Plaques were lifted in duplicate using 80 mm nitrocellulose discs impregnated with 10 mM isopropyl-β-D-thiogalactoside (IPTG). Screening followed the protocol of Sambrook et al. (1989) using anti-P. salmonis rabbit serum. Immunoreactive plaques were picked and rescreened until pure cultures were obtained. Lambda clones were then amplified and the pBluescript phagemid excised into E. coli.

Screening of the P. salmonis expression library with high titre anti-P. salmonis rabbit serum identified several strongly immunoreactive plaques. These plaques were picked and rescreened until pure and were confirmed to contain inserts. Initial attempts to excise the clones into E. coli from the lambda clones were unsuccessful which suggested the clones may encode products toxic to E. coli. Restriction fragment length analysis using frequently cutting enzymes suggested that all clones contained a common region of DNA. The clones contained a 5 kb insert (Example 1).

Genomic DNA from all the lambda clones, P. salmonis, CHSE-214, and vector plasmid DNA was analyzed by DNA dot blotting using insert DNA from one clone (Clone pB12) as the probe. Hybridization revealed that the pB12 insert was of P. salmonis origin. The pB12 insert also hybridized with all other immunoreactive lambda clone samples indicating that all the inserts encoded an overlapping fragment of P. salmonis DNA.

e. DNA Sequence Analysis of Clone pB12

DNA sequence analysis of clone pB12 (Example 1) identified 4 complete ORF's within the 4,983 bp insert and 1 partial ORF (Example 1). The predicted amino acid sequences of these ORF's was subjected to homology searches using alignment programs (eg. BLAST2 and FASTA3). No significant matches were found when searching for DNA sequence homology to the pB12 insert.

The 499 bp 'alr ORF (Example 1) was predicted to encode a 176 residue (res.) protein fused to the N-terminus of LacZ. The predicted molecular weight (m.w.) of the LacZ-'Alr fusion is 22.2 kDa. The predicted 'Alr ORF amino acid sequence shares 44% identity and 63% similarity with C-terminal portions of known alanine racemase enzymes from Klebsiella aerogenes (GenBank AAC38140), Salmonella typhimurium (GenBank A29519), and E. coli (GenBank BAA36048).

A 732 bp ORF (bax; Example 1) was predicted to encode a 243 res., 27.6 kDa protein. Both FASTA3 and BLAST2 only identified low scoring similarity (33% identical, 49% similar) between the central 187 amino acid region of the bax ORF and a 274 res. uncharacterized, hypothetical protein in E. coli K12 (BAX; GenBank AAB18547).

A 1368 bp ORF (radA; Example 1) was predicted to encode a 456 res., 49.4 kDa protein. A high degree of amino acid homology was found over the entire length of the radA ORF and RadA DNA repair enzymes from a variety of bacteria. P. salmonis RadA is most homologous to RadA of Pseudomonas aeruginosa (SwissProt P96963) with 62% identity and 77% similarity. P. salmonis RadA also exhibits 59% identity and 75% similarity to E. coli RadA (SwissProt P24554).

A 486 bp ORF (ospA; Example 1), immediately following radA, was predicted to encode a 162 res., 17.7 kDa protein with amino acids 21-162 having substantial sequence similarity with the mature chain of the rickettsial 17 kDa genus common antigen. The predicted 17 kDa antigen was up to 41% identical and 62% similar to the 17 kDa protein antigens of R. prowazekii (SwissProt GI 12704), Rickettsia japonica (SwissProt Q52764), Rickettsia rickettsii (SwissProt P05372), and Rickettsia typhi (SwissProt P22882). The 17 kDa protein of rickettsiae is translated as a precursor protein containing a 20 amino acid signal peptide. During processing the signal peptide is removed and the N-terminal cysteine residue is lipid-modified to form the mature protein. The first 21 amino acids of the P. salmonis OspA protein are predicted to be a signal peptide and contain a bacterial lipidation pattern as well.

The final 717 bp ORF (tnpA; Example 1) was predicted to encode a 239 res., 27.7 kDa protein. This ORF is flanked by a perfect 288 bp direct repeat. Amino acid similarity searches returned strong matches between the tnpA ORF and a variety of transposases. The closest match was a transposase (GenBank U83995) in a Porphyromonas gingivalis insertion element, IS195, with 47% identity and 65% similarity (Lewis and Macrina, 1998).

f. Identification of the ospA ORF as the 17 kDa Antigen

Rabbit antibodies raised against 10-mer and 20-mer synthetic peptides of this region reacted with an immunoreactive product in P. salmonis around the 17 kDa predicted mass of the ospA ORF product (Example 2). Expression of the 17 kDa antigen was induced in clone pBC-17kDa and was recognized by rabbit serum against the synthetic peptides (Example 2). Serum from coho salmon fry that had survived a challenge with P. salmonis also recognized the induced 17 kDa product (Example 2). These data confirm that the ospA ORF encodes the immunoreactive 17 kDa OspA antigen.

g. Optimization of the ospA ORF for E. coli Expression

The coding sequence of ospA was optimized using codons used frequently by E. coli (Example 3). Six overlapping oligonucleotides representing the optimized ospA gene were synthesized using standard phosphoamidite method. The gene was assembled using 2 successive PCR reactions with the oligonucleotides and the full length product was cloned into an appropriate cloning vector. DNA sequence of the optimized ospA gene was verified by sequence analysis using an automated sequencer. Production of the OspA protein from the optimized ospA gene was confirmed upon subcloning the optimized ospA gene to the pET21 (+) (Novagene) expression vector and inducing expression using the T7 promoter (Example 3).

h. Description of the Fusion Protein Constructs

The level of OspA production from the optimized ospA gene was still relatively low. It is well known to persons skilled in the art that fusion partners can aid in increasing the level of production of proteins. We constructed both N- and C-terminal fusions (Examples 4 & 5) with the ospA gene. In our examples we show that some fusions resulted in increased production of the OspA-fusion with the N-terminal fusion partner being more favourable than the C-terminal fusion partner. It is possible that presence of a signal peptide on the N-terminus of OspA may hamper high level production of OspA. Therefore, the N-terminal fusion partner may increase OspA production by masking the signal peptide. Similar increases in OspA production may be obtained from deletion of the region of the ospA gene that encodes the signal peptide.

TCE's tt P2 (SEQ ID 17) and MVF (SEQ ID 18) were synthesized as oligonucleotides using codons optimized for high level expression in E. coli. The epitope coding regions of the MVF and tt oligonucleotides were flanked by BamH I, Nde I and Vsp I, Hind III restriction endonuclease sites and primer binding sites for subsequent PCR amplification and subcloning. The MVF and tt P2 oligonucleotides were converted to double stranded DNA and amplified by PCR using standard conditions (Giovannoni, 1991) and cloned into pBC-V using BamH I and Hind III restriction endonuclease sites to create pBC-MVF and pBC-ttP2. Vector pBC-V is a variant of pBC KS(+) that lacks Vsp I restriction endonuclease sites at 925 and 984 bp. pBC KS(+) was digested with Vsp I, single stranded ends were filled in using Klenow fragment, and blunt end ligation was performed to create pBC-V.

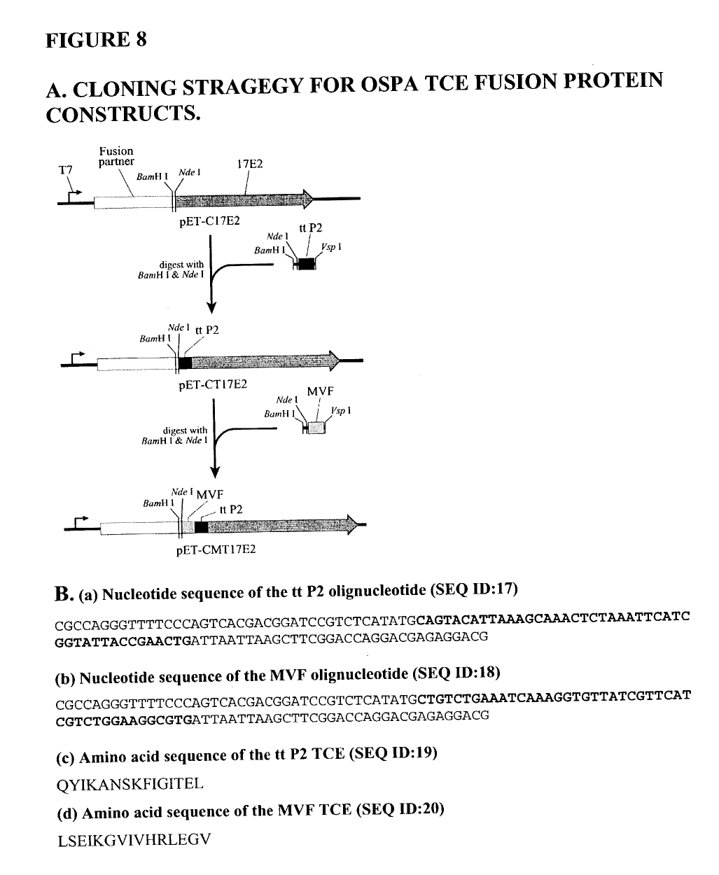

The BamH I and Vsp I fragments of pBC-MVF and pBC-ttP2 were separately subcloned into the BamH I and Nde I sites of pET-C17E2 (FIG. 8). This subcloning step placed the TCE's in frame between ospA and the N-terminal fusion partner to create pET-CM17E2 and pET-CT17E2 (FIG. 8). Ligation of the Vsp I and Nde I cohesive ends destroyed the respective restriction sites while an Nde I site was encoded in the 5′-terminal region of the TCE insert to allow subsequent ligation of inserts in frame and upstream of the TCE using BamH I and Nde I (FIG. 8).

A third construct encoding both TCE's was created by subcloning the BamH I and Vsp I fragment of pBC-MVF into the BamH I and Nde I sites of pET-CT17E2 to create pET-CMT17E2 (FIG. 8).

### EXAMPLES

The following examples are included to demonstrate preferred embodiments of the invention, and it will be appreciated by those skilled in the art, in light of this disclosure, that many changes can be made in the specific embodiments disclosed without departing from the scope of the invention.

1. Sequence Analysis of P. salmonis Insert Producing Immunoreactive Material

A directional deletion library of P. salmonis clone pB12 was constructed to facilitate sequence analysis. Exo III and S1 nuclease were used to construct double-stranded nested deletions in the direction of lacZ. Restriction endonucleases EcoR I and Sac I were used to generate opposing overhangs protecting the vector from Exo III digestion. Upon ligation and screening, 32 deletion clones were selected that represented the entire insert and differed in size by 100-500 bp.

Double stranded plasmid DNA samples were sequenced using a combination of dye primer and dye termination. Sequencing reactions were analyzed using an automated DNA sequencer. Sequence data were assembled and analyzed using commercially available computer software packages.

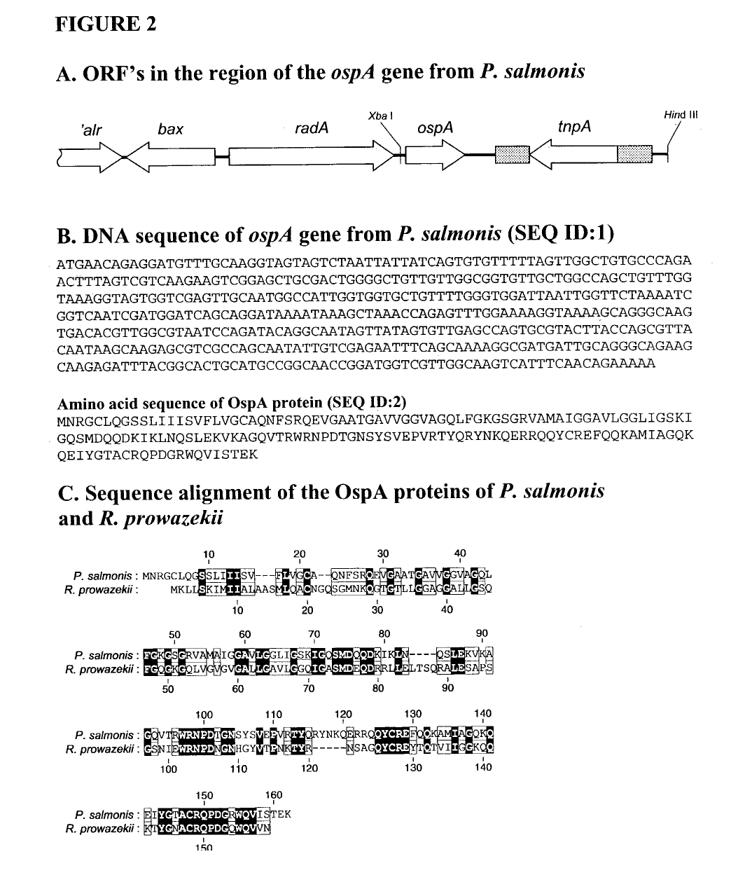

DNA sequencing of pB12 Exo III/S1 nuclease deletion clones revealed that the insert was 4,983 bp. Coding predictions identified 4 intact ORF's and 1 partial ORF creating a fusion in frame with LacZ (FIG. 2). The predicted ORF's were subjected to BLAST2 (Altschul, et al., 1997) and FASTA3 (Pearson, 1998) analysis to determine if any similar sequences were known (FIG. 2).

2. Identification of the ospA ORF as the Source of OspA

Residues 110-129 of the 17 kDa antigen encoded by the predicted ospA ORF were predicted to be a B cell epitope by the Jameson-Wolf method (Jameson and Wolf, 1988). Antibodies were generated in New Zealand white rabbits against 10 and 20 amino acid synthetic peptides (SEQ ID:15; SEQ ID:16) representing amino acids 110-129 of the predicted OspA amino acid sequence (SEQ ID:2). Peptides were glutaraldehyde conjugated to for 1 h at 4° C. in a 10 ml reaction volume with 500 μg/ml keyhole limpet hemocyanin and 1% glutaraldehyde. For the primary immunization, rabbits received 250 μg of conjugated peptide mixed 1:1 with Freund's complete adjuvant. Each rabbit was boosted three times at 2 week intervals with 250 μg of conjugated peptide per boost mixed 1:1 with Freund's incomplete adjuvant.

TABLE 2

Synthetic polypeptides used to generate polyclonal rabbit antibodies against OspA.

| | |
|---|---|
| Peptide | Sequence |

| | | |
|---|---|---|
| 10 mer | Pro-Val-Arg-Thr-Tyr-Gln-Arg-Tyr-Asn-Lys | (SEQ ID:15) |
| 20 mer | Pro-Val-Arg-Thr-Tyr-Gln-Arg-Tyr-Asn-Lys-Gln-Glu-Arg-Arg-Gln-Gln-Tyr-Cys-Arg-Glu | (SEQ ID:16) |

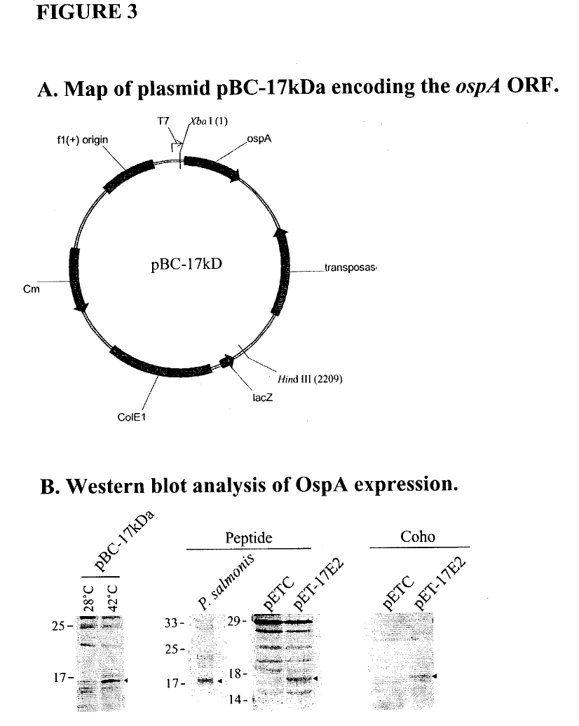

The 17 kDa antigen ospA ORF was subcloned into pBC(+) under control of the T7 promoter. The Xha I/Hind III fragment of clone pB12 was ligated with Xba I/Hind III digested pBC(+) to generate clone pBC-17 kDa. Induction of the T7 promoter by shifting growth temperature to 42 C. resulted in expression of a 17 kDa protein observed by Coomassie staining of whole cell lysates of induced clone pBC-17kDa SDS-PAGE samples (FIG. 3). Western blot analysis of whole cell lysates of P. salmonis and pBC-17 kDa with rabbit antibodies generated against synthetic peptides of OspA reacted with a 17 kDa protein in both P. salmonis and the induced sample of pBC-17 kDa confirming the ospA ORF as the source of then translated OspA protein (FIG. 3).

3. Synthesis & Cloning of Optimized ospA Gene

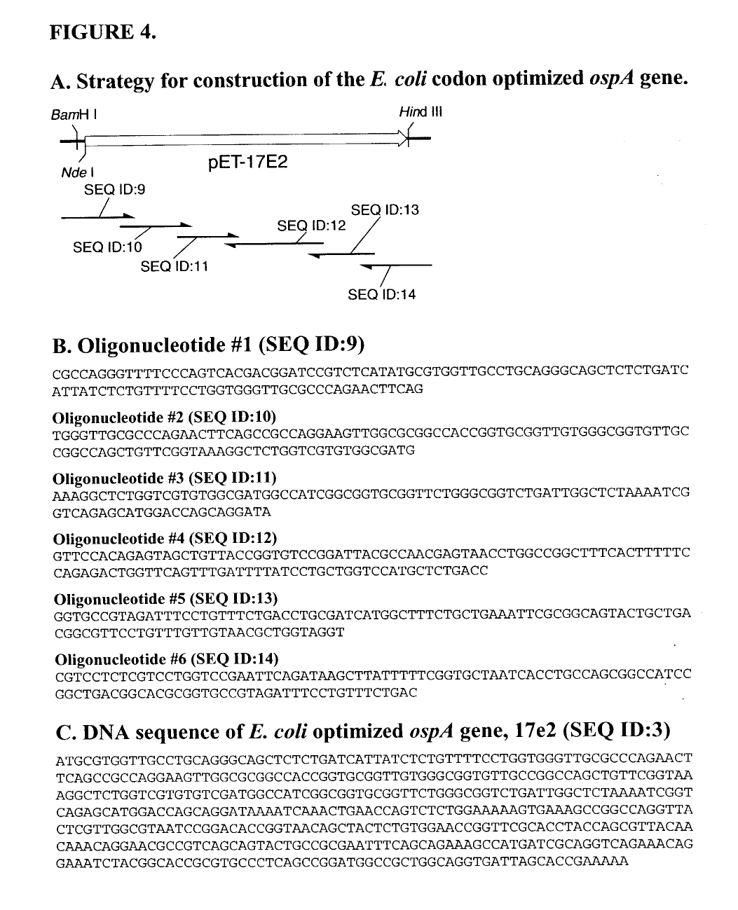

A nucleic acid molecule was designed to encode the OspA protein precursor (OspA including signal peptide). This nucleic acid was constructed by PCR using 6 overlapping oligonucleotides (SEQ ID:9, SEQ ID:10, SEQ ID:11, SEQ ID:12, SEQ ID:13, and SEQ ID:14). Synthesis of ospA gene was done by three subsequent PCR using the six synthetic overlapping oligonucleotides (FIG. 4A & FIG. 4B). PCR-1 involved overlapping oligonucleotides SEQ ID:11, SEQ ID:12 (0.05 pmol/μl each) and SEQ ID:10, SEQ ID:13 (0.25 pmol/μl each). Product of PCR-1 (1 μl) was used as a template in PCR-2 using oligonucleotides SEQ ID:9 and SEQ ID:14 as primers (0.25 pmol/μl). Both PCR were performed using Taq I polymerase (Boehringer), supplied buffer and deoxynucleotide triphosphates (dNTP) (Amersham Pharmacia). Temperature cycling was as follows: PCR-1 & 2:

92 C. 30 sec., 55 C. 30 sec., 72 C. 30 sec., 1 cycle

92 C. 30 sec., 70 C. 30 sec., 72 C. 30 sec., 29 cycles

Product of PCR2 (FIG. 4C) was cloned into plasmid vector pBC(+) as a BamH I-Hind III fragment resulting to pBC-17E2. DNA sequence of the insert was verified by DNA sequencing using methods known to those skilled in the art. The DNA fragment of pBCKS-17E2 carrying optimized ospA gene was than cloned to pET21(+) as a Nde I-Hind III DNA fragment resulting to pET-17E2.

4. Expression of Optimized OspA Antigen With N-Terminal Fusion Partner

A. Expression using T7 Promoter System

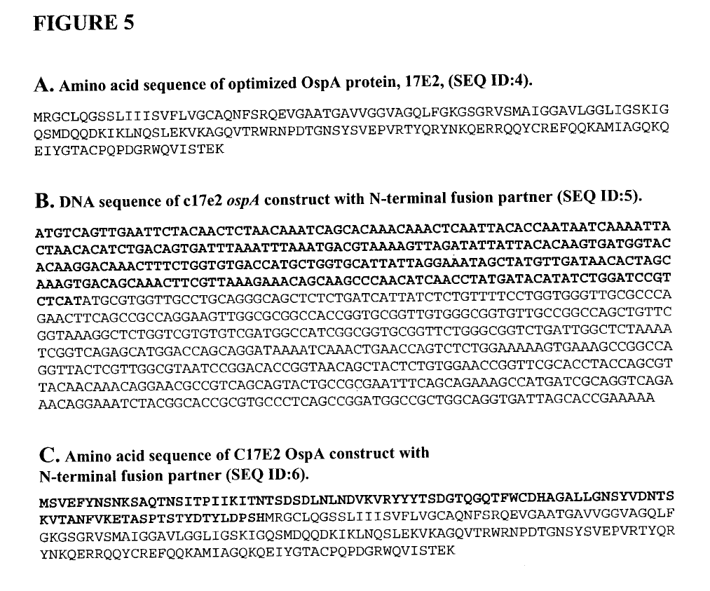

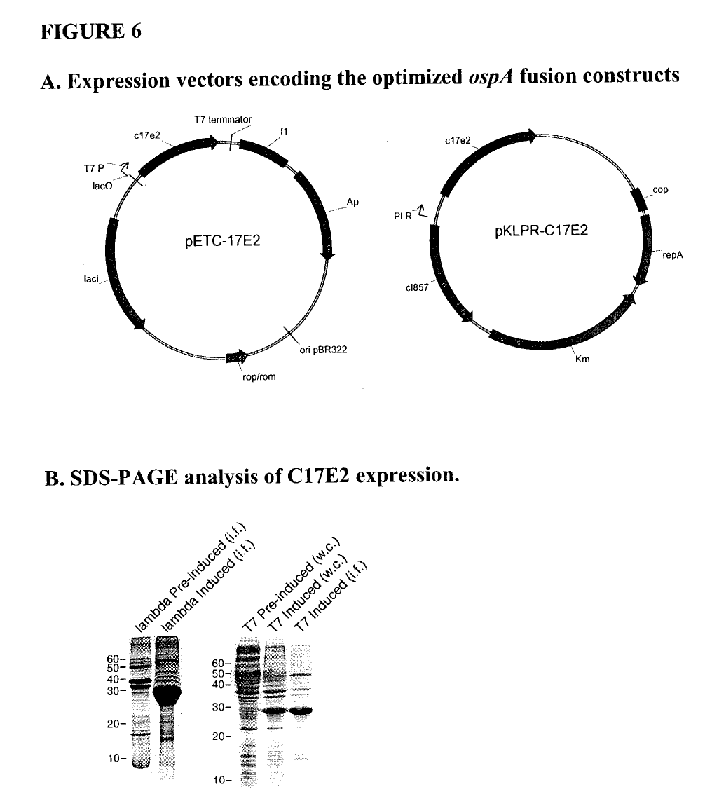

DNA fragment of pBCKS-17E2 carrying optimized ospA gene was cloned, using methods known to one skilled in the art, to pETC (Microtek International) resulting to pETC-17E2 as a BamHI-HindIII fragment carrying ospA fused to a desired fusion partner under control of T7 promoter (FIG. 5, FIG. 6A).

Strain E. coli BL21 [E. coli B, F-, ompT, hsdS (rₛ-, m₂-), gal, dcm] (Pharmacia) carried the recombinant expression plasmid pETC-17E2 and helper plasmid pGP1-2 (Tabor and Richardson, 1985). Expression experiment was performed in 4 L flask. During the growth phase, the culture was grown in Terrific Broth (TFB) with agitation (˜300 RPM) at 28-30 C. to late log phase. Then cells were diluted with an equal volume of fresh TFB media and growth continued at 42 C. 3-6 hours. Product was accumulated inside cells as insoluble aggregates of protein. Cells from 1 ml of culture were sedimented in a microcentrifuge, washed with water, resuspended in 1 ml of water and disrupted by sonication. Insoluble material was sedimented, washed with water and analyzed by 15% SDS-PAGE as is known to one skilled in the art (FIG. 6B).

B. Expression using Lambda Promoter System

DNA fragment of pETC-17E2 carrying fused optimized ospA gene was subcloned, using methods known to one skilled in art, to pKLPR-8 (Microtek International 1998 Ltd.) resulting in pKLPR-C17E2 as a Xba I-Kpn I fragment carrying the ospA fusion under control of phage lambda promoter. Plasmid also carries repressor gene CI875 of the lambda promoter (FIG. 5).

Strain E. coli BL21 [E. coli B, F-, ompT, hsdS (rₛ-, mₛ-), gal, dcm] (Pharmacia) carried the recombinant expression plasmid pKLPR-C17E2 (FIG. 6A). During the growth phase, the culture was grown in TFB with agitation (300 RPM) at 28-30 C. to late log phase. Then cells were diluted with an equal volume of fresh TFB media and growth continued at 42 C. 3-6 hours. Product was accumulated inside cells as insoluble aggregates of protein. Cells from 1 ml of culture were sedimented in a microcentrifuge, washed with water, resuspended in 1 ml of water and disrupted by sonication. Insoluble material was sedimented, washed with water and analyzed by 15% SDS-PAGE as is known to one skilled in the art (FIG. 6B).

5. Expression of Optimized OspA Antigen With C-Terminal Fusion Partner

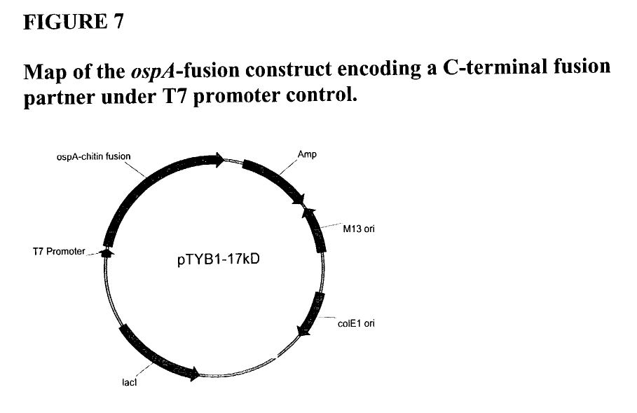

The P. salmonis ospA ORF was subcloned into the Impact CN Expression System (New England Biolabs) to add a C-terminal fusion partner containing a self-cleaving spacer region and chitin binding domain to aid in purification and antibody generation of OspA (FIG. 7).

The ospA ORF was PCR amplified from clone pB12 using custom primers (Table 3) designed to incorporate Nde I and Sap I restriction enzyme cleavage sites onto the 5′ and 3′ ends of the ospA ORF. The ospA PCR product was digested with Nde I and Sap I restriction enzymes and ligated with the pTYB1 vector (NEB) of the Impact CN system digested with Nde I and Sap I to create the OspA fusion construct, pTYB1-17kDa (FIG. 7). Positive clones were identified by screening Kpn I and Nde I digests of plasmid preps from potential positive clones by agarose gel electrophoresis. Positive clones were confirmed to contain the ospA ORF in frame with the chitin binding domain by DNA sequence analysis.

TABLE 3

Oligonucleotide primers used during construction of pTYB1-17kDa. Bold
nucleotides are not homologous to the template ospA ORF.

| | |
|---|---|
| Primer | Sequence |

| | | |
|---|---|---|
| Forward | 5′-GAG AGA ACA TAT GAA CAG AGG ATG TTT GCA AGG-3′ | (SEQ ID:7) |
| Reverse | 5′-GCC ATA AGC TCT TCC GCA TTT TTC TGT TGA AAT GAC TTG C-3′ | (SEQ ID:8) |

6. Salmonid Antibody Response to OspA-fusion Vaccine

Coho salmon antibody response to the OspA with N-terminal fusion partner vaccine candidate (Example 4) was assayed by enzyme linked immunosorbant assay (ELISA). Coho salmon fry (125 per group; ˜15 g mean weight) were each injected intraperitoneally (IP) 0.2 ml of a formalin inactivated (1 ml/L) adjuvanated (Microgen™) vaccine (5:1 vaccine:adjuvant) containing 50 μg of total protein purified as the insoluble fraction from E. coli BL21 expressing the ospA fusion construct pET-C17E2 (Example 4). A control group of fish received 0.2 ml of adjuvant diluted with saline 5:1. A second control group was comprised of non-vaccinated salmon.

Four weeks post-immunization, 5 fish from each group were bled from the caudal vein, kept on ice, blood was pooled for each group and serum was collected by centrifugation of pooled blood at 5,000 rpm for 20 min in a clinical centrifuge. ELISA plates were coated with 10 μg of C17E2 protein in 100 μl of coating buffer (Tris buffered saline (TBS), pH 7.5, 0.5% Tween-20). Plates were covered with parafilm and incubated at 4 C. overnight. Coating solution was removed and wells were blocked with 200 μl of Tween-TBS with 3% bovine serum. Plates were washed 3 times with Tween-TBS. Fish serum from each group was serially diluted in Tween-TBS with 3% bovine serum and added to wells. Plates were then incubated at 15 C. for 1 h and then washed 3 times with Tween-TBS. Second antibody, a mixture of 2 monoclonal antibodies (mAb) against salmon immunoglobulin, IPA2C7 (dil. 1/100) and Beecroft (dil. 1/500), were diluted in Tween-TBS with 3% bovine serum, added to plates and incubated at room temperature for 1 h. Plates were washed 3 times with TBS-Tween. Third antibody, alkaline phosphatase conjugated goat anti-mouse IgG₁ (dil. 1/2000), was added to plates and incubated at room temperature for 1 h. Plates were washed 3 times. The ELISA was developed with 100 μl of 1 mg/ml para-nitrophenyl phosphate in alkaline phosphatase buffer and incubated at room temperature overnight and absorbance at 405 nm was measured spectrophotometrically.

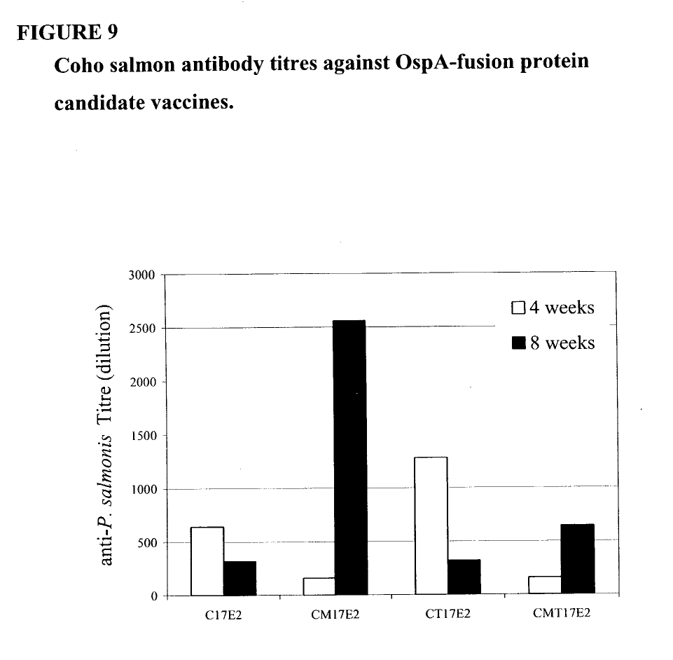

Antibody titres were defined as the maximum serum dilution that resulted in a signal corresponding to 3 times the background obtained with the diluent vaccinated serum group at a dilution of 1:320. Background serum was pooled from coho salmon vaccinated with adjuvant alone at each time point. The results indicate that all OspA fusion protein constructs are capable of eliciting an antibody response in immunized coho higher than the response obtained with adjuvant alone. The highest antibody responses were found in coho salmon immunized with OspA fusion proteins containing promiscuous TCE's (FIG. 9).

7. Salmonid Lymphocyte Response to OspA-fusion Vaccine

The lymphocyte response to the OspA-fusion protein vaccine constructs was measured using the lymphocyte proliferation assay.

Isolation of lymphocytes. Atlantic salmon that had been vaccinated 4 weeks prior with 0.2 ml of each OspA fusion protein vaccine were euthanized with an overdose of marinil and their head kidneys were aseptically harvested and immediately placed in 5 ml of cold MEM-10 (10% fetal bovine serum; Life Technologies) on ice. All subsequent manipulations were conducted on ice. Cells were dissociated by repeated passage through a 5 ml syringe. The tissue suspension was placed in a 15 ml tube and 7 ml of additional MEM-10 were added. Tissue fragments were allowed to settle out of solution for 10 min. Cells suspended in the media were collected and layered on 4 ml of 51% Percoll (10 ml 10×HBSS, 51 ml Percoll, made up to 100 ml with H₂O). The step gradient was centrifuged for 30 min at 400×g, 4° C. Lymphocytes were collected from the MEM-10/Percoll interface. Lymphocytes were centrifuged and washed once in MEM-10 and resuspended in 1 ml MEM-10. The numbers of viable cells was determined using Trypan blue (0.4%; Sigma) staining. Cells were diluted to a final concentration of 5×10⁶ cells/ml with MEM-10.

Lymphocyte proliferation assay. Isolated lymphocytes were added to 96 well cell culture plates with 5×10⁵ cells/well (100 μl vol.). OspA fusion protein C17E2 was added as a stimulating antigen (2 μg/well) and cells were incubated for 6 days at 17° C. Lymphocyte proliferation was determined spectrophotometrically using WST-1 (4-[3-(4-iodophenyl)-2-(4-nitrophenyl)-2H-5-tetrazolio]-1,3-benzene disulfonate) cell proliferation reagent (Roche Molecular Biochemicals). WST-1 allows colorimetric quantification of cell proliferation based on cleavage of WST-1 by mitochondrial dehydrogenases in viable cells. WST-1 (10 μl) was added to each well and plates were incubated at 17° C. until sufficient colour development prior to absorbance measurement at 450 nm with a reference wavelength of 630 nm. Bacterial lipopolysaccharide (LPS) (100 μg/ml) and conconavalin A (ConA) (50 μg/ml) were used as B and T lymphocyte mitogens for positive controls.

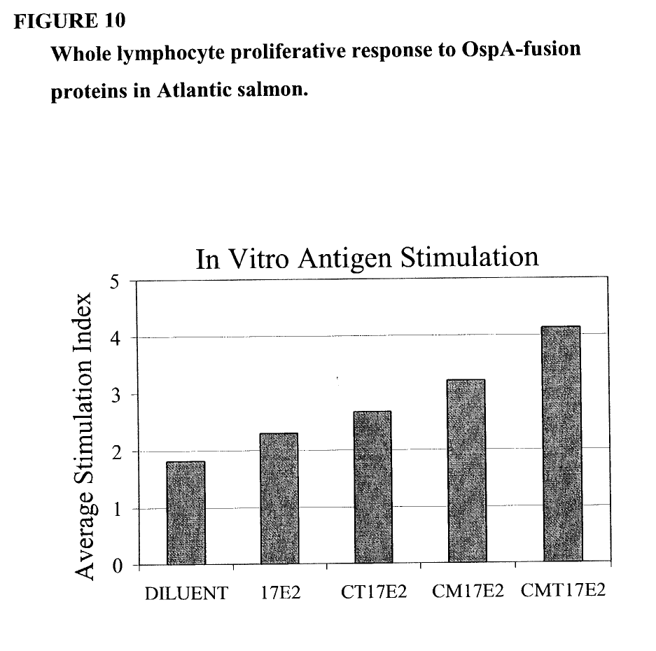

The degree of lymphocyte stimulation was determined by calculating the stimulation index for each sample of lymphocytes exposed to antigen (FIG. 10). Stimulation index was calculated by dividing the average absorbance of lymphocyte samples presented with stimulating antigen by the average absorbance of lymphocytes presented with no antigen (FIG. 10). The results indicate the addition of promiscuous TCE's to the OspA fusion protein candidate enhance the proliferative lymphocyte responses of salmon vaccinated with the TCE-encoding vaccines against OspA (FIG. 10).

8. Protection of Immunized Salmonids Against P. salmonis Challenge

OspA fusion proteins were purified as inclusion bodies from E. coli BL21 and protein concentrations were determined using the BCA protein assay (Pierce). The relative percentages of the OspA fusion proteins within each preparation were determined by SDS-PAGE analysis and quantification of the fusion protein bands using a Gel Documentation system and AlphaEase software. Each protein sample was fixed by the addition of formalin (1 ml/L) and incubation with shaking at 15° C. for 24 hr. Each protein solution was added aseptically to diluent (oil in water adjuvant) to obtain a final target protein concentration of 250 mg/L.

Coho salmon (˜15 g) were anaesthetized (1 ppm metomidate hydrochloride), fin clipped for group identification, and intraperitoneally injected with 0.2 ml of vaccine with 60 fish per group. There were 6 groups in total: C17E2, CT17E2, CM17E2, CMT17E2, CM 17E2 plus CT17E2 (1:1), and an adjuvant control. Salmon were held for 8 weeks in freshwater at 8.5° C. post-vaccination.

All vaccinated coho were anaesthetized (1 ppm Marinil) and IP injected with 0.1 ml of P. salmonis infected CHSE-214 cell culture supernatant (˜10⁶ TCID₅₀/ml). Salmon were maintained in freshwater at 13° C. post-challenge and mortalities were logged. External and internal observations along with PCR of kidney and central liver sections using P. salmonis 16S rRNA primers (Giovannoni, 1991; Marshall, et al., 1998) were performed for confirmation of mortality.

RPS is calculated to generate a numerical value representing the level of protection elicited by a vaccine. In general, RPS is calculated as a ratio of the cumulative mortality of a test group to the cumulative mortality of an unvaccinated group. RPS=[1−(% mortality of test group÷% mortality of control group)]×100%.

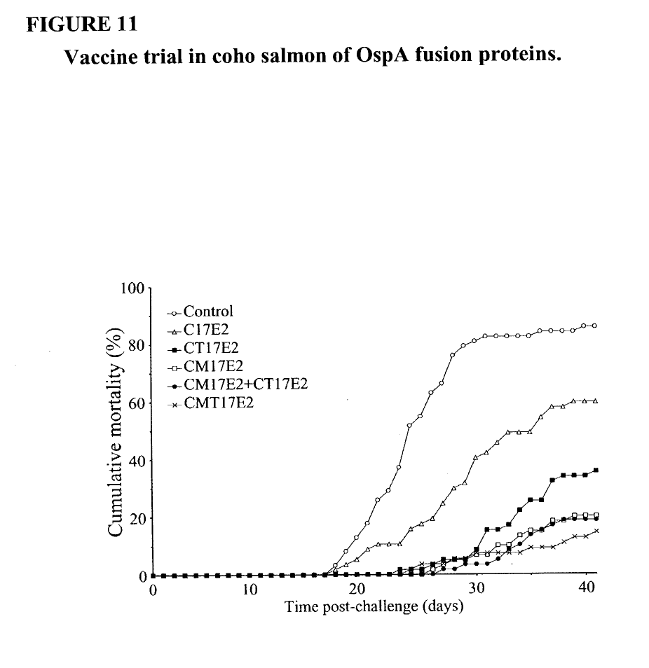

Mortalities in the TCE OspA construct vaccinated groups began 7-10 days after the control group (FIG. 11). Cumulative mortality reached 85.5% in the control group (FIG. 11). The C17E2 vaccinated group reached 59.6% cumulative mortality, 30.2% RPS (FIG. 11). The CT17E2 vaccinated group reached a cumulative mortality of 35.6%, 58.4% RPS (FIG. 11). CM17E2 vaccinated salmon reached 20.0%, 76.6% RPS (FIG. 11). Salmon vaccinated with a 1:1 mixture of CM17E2 and CT17E2 reached 18.6% cumulative mortality giving a 78.2% RPS (FIG. 11). The lowest mortality was observed in the CMT17E2 vaccinated group, with only 14.5% cumulative mortality and an 83.0% RPS (FIG. 11).

The results indicate that adjuvant controls (o) had severe mortalities (>80%) and the CMT17E2 vaccinates (x) were significantly protected with only 14.5% mortality (FIG. 11).

1. A method of incorporation of lymphocyte T cell epitopes and lymphocyte B cell epitopes or one of lymphocyte T cell epitopes or lymphocyte B cell epitopes into one or more chimeric fusion proteins in fish.

2. The method of claim 1 wherein the TCE's (T cell epitopes) are further defined as highly immunogenic promiscuous TCE's.

3. The method of claim 2, implemented by a vaccine.

4. The method of claim 1 wherein the fusion protein is comprised of one of OspA, an OspA lipoprotein, its variants, its non-lipidated form, or antigenic peptides derived or synthesized thereof.

5. The method of claim 2, wherein the fish is a selected salmonid.

6. The method of claim 4, wherein the fish is a selected salmonid.

7. The method as defined in claim 4, implemented by a vaccine.

8. The method of claim 5 wherein the said vaccine or variants thereof are encapsulated in or absorbed in or adsorbed to or are in the form of an insoluble polymeric matrix.

9. The method of claim 6 wherein the said vaccine or variants thereof are encapsulated in or absorbed in or adsorbed to or are in the form of an insoluble polymeric matrix.

10. The method of claim 6 where the vaccine is formulated with an adjuvant.

11. The method of claim 1, wherein fusions of at least one of the DNA sequence encoding T cell epitopes or B cell epitopes, fragments or synthetic oligonucleotides thereof or of DNA sequence homologues of T cell epitopes or B cell epitopes, or fragments or synthetic oligonucleotides derived thereof is incorporated thereby incorporating one or more chimeric fusion proteins in fish.

12. The method of claim 2 wherein fusions of at least one of a DNA sequence corresponding to that of TCE's fragments or synthetic oligonucleotides thereof or of DNA sequence homologues of TCE's, or fragments or synthetic oligonucleotides derived thereof is incorporated thereby incorporating one or more chimeric fusion proteins in fish.
